(19) United States
(12) Patent Application Publication
(10) Pub. No.: US 2026/0133173 A1
(43) Pub. Date: May 14, 2026

(54) METHOD AND SYSTEM FOR ASSESSING SAFETY RISK LEVEL OF FEED IN CATTLE AND SHEEP BREEDING

(71) Applicant: Ningxia University, Yinchuan (CN)

(72) Inventors: Guosheng Xin, Yinchuan (CN); Yumin Nie, Yinchuan (CN); Wei Han, Yinchuan (CN); Li Hu, Yinchuan (CN); Yanming Wang, Yinchuan (CN)

(21) Appl. No.: 19/376,730

(22) Filed: Oct. 31, 2025

(30) Foreign Application Priority Data
Nov. 8, 2024 (CN) 2024115877912

(51) Int. Cl. G01N 33/02

(52) U.S. Cl. CPC G01N 33/02

(57) ABSTRACT

A method and a system for assessing a safety risk level of a feed in cattle and sheep breeding are provided. The method includes: identifying accurately potential chronic toxic substances in the feed for cattle and sheep through sample testing and data analysis; constructing, according to the potential chronic toxic substances, a biological enrichment abnormal index and a metabolic pathway deviation index, an accumulation effect model of the chronic toxic substances in cattle and sheep bodies; assessing by the system health risks of the chronic toxic substances according to the accumulation effect model, and classifying the safety risk level of the feed as one of a high-risk level, a medium-risk level, and a low-risk level; and formulating corresponding management measures. When the safety risk level of the feed is medium, the system further dynamically adjusts the safety risk level to respond in real time to potential high-risk scenarios.

### CROSS-REFERENCE TO RELATED APPLICATION

This application claims priority to Chinese Patent Application No. 202411587791.2, filed Nov. 8, 2024, which is herein incorporated by reference in its entirety.

### TECHNICAL FIELD

The disclosure relates to the field of cattle and sheep breeding technology, and more particularly to a method and system for assessing a safety risk level of feed in cattle and sheep breeding.

### BACKGROUND

The safety risk level assessment of feed in cattle and sheep breeding refers to a process of grading and evaluating the potential safety risks that the cattle and sheep feed may bring during the breeding process through a systematic approach. This assessment aims to identify potential safety hazards that may arise in various stages of feed production, storage, transportation, and use, and to analyze the impact of these risks on the health of cattle and sheep as well as the quality of the final agricultural products, thereby providing a scientific basis for breeding enterprises and regulatory authorities to help reduce the safety risks brought by the feed.

During the assessment process, experts usually take into account a variety of factors, such as whether the sources of feed ingredients are compliant, the rationality of nutritional components, and the levels of harmful substances (such as mycotoxins, heavy metals, and pesticide residues). The assessment results are generally divided into different risk levels, such as low, medium, and high risks, so that breeding farms can take appropriate measures accordingly. For example, high-risk feed may require stricter testing or improved feed formulas, while low-risk feed can be used safely. This can help ensure the healthy development of the livestock industry, as well as the quality of cattle and sheep products and the food safety of consumers.

The deficiencies in the related art are as follows.

The current methods for assessing the safety of cattle and sheep feed mainly focus on acute toxicity and neglect the cumulative effects of low-concentration chronic toxic substances after long-term intake. Trace amounts of heavy metals, mycotoxins, and antibiotic residues in feed, although not posing an obvious threat to health in the short term, will gradually accumulate in cattle and sheep and may eventually lead to chronic diseases or organ damage. However, the existing assessment system lacks a precise predictive model for the accumulation effect of chronic toxic substances after intake by cattle and sheep, and is unable to effectively predict the long-term impacts of these chronic toxic substances. In addition, the risk-level classification is usually static, lacking a dynamic adjustment mechanism and refined management tools, making it difficult to respond in a timely manner to the high-risk evolution of chronic toxic substances under specific conditions, which leads to safety hazards.

### SUMMARY

The disclosure aims to provide a method and a system for assessing a safety risk level of a feed in cattle and sheep breeding to address the shortcomings in the background technology.

In order to achieve the above purposes, the disclosure provides the following technical solutions. A method for assessing a safety risk level of a feed in cattle and sheep breeding includes the following steps:
S1, identifying different low-concentration chronic toxic substances in the feed for cattle and sheep through sample testing and data analysis, where the different low-concentration chronic toxic substances include heavy metals, mycotoxins, antibiotic residues, and environmental pollutants;
S2: obtaining, based on the different low-concentration chronic toxic substances, a biological enrichment abnormal index and a metabolic pathway deviation index of each of the different low-concentration chronic toxic substances, constructing an accumulation effect model of the different low-concentration chronic toxic substances in cattle and sheep bodies, and predicting accumulation levels of the different low-concentration chronic toxic substances after intake by cattle and sheep;
S3: assessing, according to the accumulation effect model, potential health risks of the different low-concentration chronic toxic substances after the intake by cattle and sheep to obtain assessment results; classifying, according to the assessment results, the safety risk level of the feed as one of different risk levels including a high-risk level, a medium-risk level, and a low-risk level; and formulating corresponding management measures and feed usage restrictions for the different risk levels; and
S4: when the safety risk level of the feed is classified as the medium-risk level, further analyzing the potential health risks of the different low-concentration chronic toxic substances after the intake by cattle and sheep within a fixed time period to obtain analysis results, and dynamically adjusting, according to the analysis results, the safety risk level of the feed.

In an embodiment, in step S2, the obtaining, based on the different low-concentration chronic toxic substances, a biological enrichment abnormal index and a metabolic pathway deviation index of each of the different low-concentration chronic toxic substances includes: obtaining the biological enrichment abnormal index, including:

defining factors affecting a biological enrichment process of a toxic substance of the different low-concentration chronic toxic substances in the cattle and sheep bodies as nodes in a Bayesian network, where the factors include: an intake I, an absorption rate A, a metabolism rate M, and an excretion rate E, and marking a concentration accumulation level of the toxic substance as C; setting a conditional probability table for each of the nodes to describe a variable value and a probability of the variable value under a given parent node condition; solving, according to a structure of the Bayesian network and the conditional probability table, a marginal probability of the concentration accumulation level through marginalization as follows: P(C)=Σ_{I} Σ_{A} Σ_{M} Σ_{E} P(C|A,M,E)·P(A∥)·P(I)·P(M)·P(E), where P(C) represents an accumulation probability distribution of the toxic substance in vivo; and setting a normal accumulation range as Cₙₒᵣₘₐₗ, and calculating the biological enrichment abnormal index through the following formula: BA=P(C>Cₙₒᵣₘₐₗ)=1−Σ_{C≤C}_{ₙₒᵣₘₐₗ} P(C), where BA represents the biological enrichment abnormal index.

In an embodiment, in step S2, the obtaining, based on the different low-concentration chronic toxic substances, a biological enrichment abnormal index and a metabolic pathway deviation index of each of the different low-concentration chronic toxic substances includes: obtaining the metabolic pathway deviation index, including:
in a dynamic metabolic network model, calculating a change in a concentration of a metabolite of metabolites Cᵢ(t) through the following formula:

$$\frac{d {C}_{i} ( t )}{d t} = {f}_{i} ( {C}_{1} ( t ) , {C}_{2} ( t ) , … , {C}_{n} ( t ) , {k}_{i} ) ,$$

where Cᵢ(t) represents the concentration of the metabolite i at time t, fᵢ represents a rate equation of metabolic reaction and is defined based on chemical reaction kinetics, kᵢ represents a reaction rate constant, and n represents a total number of the metabolites; establishing a dynamic model of the toxic substance under normal metabolic conditions, obtaining a normal metabolic parameter

$${k}_{i}^{normal}$$

and a corresponding normal metabolite concentration change

$${C}_{n}^{normal} ( t )$$

of the toxic substance, and obtaining an equation for a normal metabolic pathway as follows:

$$\frac{d {C}_{i}^{normal} ( t )}{d t} = {f}_{i} ( {C}_{1}^{normal} ( t ) , {C}_{2}^{normal} ( t ) , … , {C}_{n}^{normal} ( t ) , {k}_{i}^{normal} ) ;$$

re-measuring a metabolic parameter

$${k}_{i}^{obs}$$

and a metabolite concentration change

$${C}_{n}^{obs} ( t )$$

of the toxic substance, establishing an abnormal metabolic model, and obtaining an equation for an abnormal metabolic pathway as follows:

$$\frac{d {C}_{i}^{obs} ( t )}{d t} = {f}_{i} ( {C}_{1}^{obs} ( t ) , {C}_{2}^{obs} ( t ) , … , {C}_{n}^{obs} ( t ) , {k}_{i}^{obs} ) ;$$

calculating a metabolic deviation Dᵢ(t) at the time t through the following formula to quantify a deviation between the normal metabolic pathway and the abnormal metabolic pathway:

$${D}_{i} ( t ) = \vert {C}_{i}^{obs} ( t ) - {C}_{i}^{normal} ( t ) \vert ;$$

and calculating the metabolic pathway deviation index MP being an integral of metabolite concentration deviations over a time period through the following formula:

$$MP = \frac{1}{n} {\sum }_{i = 1}^{n} {\int }_{0}^{T} {D}_{i} ( t ) dt ,$$

where T represents a total duration of an observation period, and n represents the total number of the metabolites.

In an embodiment, step S2 further includes: constructing, based on the biological enrichment abnormal index and the metabolic pathway deviation index of each of the different low-concentration chronic toxic substances, the accumulation effect model of the different low-concentration chronic toxic substances in the cattle and sheep bodies, and predicting the accumulation levels of the different low-concentration chronic toxic substances after the intake by cattle and sheep, where the accumulation effect model is a machine learning model, specifically including:
converting the biological enrichment abnormal index and the metabolic pathway deviation index into a comprehensive feature vector for each of the different low-concentration chronic toxic substances as an input of the machine learning model;
training the machine learning model with a predicted target being to predict, based on the comprehensive feature vector for each of the different low-concentration chronic toxic substances, accumulation level value labels of the different low-concentration chronic toxic substances after the intake by cattle and sheep, and a training target being to minimize a sum of prediction errors for the different low-concentration chronic toxic substances after the intake by cattle and sheep until the sum of the prediction errors reaches convergence; and
determining, according to model output results, accumulation level values of the different low-concentration chronic toxic substances after the intake by cattle and sheep, where the machine learning model is a polynomial regression model.

In an embodiment, step S3 specifically includes:
comparing the accumulation level values of the different low-concentration chronic toxic substances after the intake by cattle and sheep with a gradient standard threshold, where the gradient standard threshold includes a first standard threshold and a second standard threshold, and the first standard threshold is less than the second standard threshold, specifically including:
   comparing the accumulation level values of the different low-concentration chronic toxic substances after the intake by cattle and sheep with the first standard threshold and the second standard threshold respectively;
when one of the accumulation level values of the different low-concentration different chronic toxic substances after the intake by cattle and sheep is greater than the second standard threshold, which indicates that the potential health risks of the different low-concentration chronic toxic substances after the intake by cattle and sheep are high, generating a first-level warning signal, classifying the safety risk level of the feed as the high-risk level, and taking restrictive measures immediately;
when one of the accumulation level values of the different low-concentration chronic toxic substances after the intake by cattle and sheep is greater than or equal to the first standard threshold and less than or equal to the second standard threshold, which indicates that the potential health risks of the different low-concentration chronic toxic substances after the intake by cattle and sheep are medium, generating a second-level warning signal, classifying the safety risk level of the feed as the medium-risk level, strengthening monitoring, and reducing a usage frequency;
when the accumulation level values of the different low-concentration chronic toxic substances after the intake by cattle and sheep are less than the first standard threshold, which indicates that the potential health risks of the different low-concentration chronic toxic substances after the intake by cattle and sheep are low, generating a third-level warning signal, classifying the safety risk level of the feed as the low-risk level, and continuing to use the feed with regular checks.

In an embodiment, step S4 specifically includes:
when the safety risk level of the feed is classified as the medium-risk level, and one of the accumulation level values of the different low-concentration chronic toxic substances after the intake by cattle and sheep generated within the fixed time period is greater than or equal to the first standard threshold and less than or equal to the second standard threshold, collecting accumulation level values of the different low-concentration chronic toxic substances after the intake by cattle and sheep generated in a subsequent fixed time period that are greater than or equal to the first standard threshold and less than or equal to the second standard threshold, establishing a data set correspondingly, calculating a mean and a standard deviation of the accumulation level values in the data set, further analyzing the potential health risks of the different low-concentration chronic toxic substances after the intake by cattle and sheep to obtain the analysis results, and dynamically adjusting, according to the analysis results, the safety risk level of the feed.

In an embodiment, the dynamically adjusting, according to the analysis results, the safety risk level of the feed includes:
when the mean of the accumulation level values in the data set is greater than or equal to a reference threshold of the mean, the standard deviation of the accumulation level values in the data set is less than a reference threshold of the standard deviation, and the mean of the accumulation level values in the data set is high with small fluctuations, upgrading the safety risk level of the feed to the high-risk level, and taking corresponding management measures;
when the mean of the accumulation level values in the data set is greater than or equal to the reference threshold of the mean, the standard deviation of the accumulation level values in the data set is greater than or equal to the reference threshold of the standard deviation, and the mean of the accumulation level values in the data set is high but the standard deviation of the accumulation level values in the data set is large, re-assessing the feed, maintaining temporarily the safety risk level of the feed at the medium-risk level, and strengthening monitoring of accumulation fluctuation;
when the mean of the accumulation level values in the data set is less than the reference threshold of the mean, the standard deviation of the accumulation level values in the data set is greater than or equal to the reference threshold of the standard deviation, and the mean of the accumulation level values in the data set is low but the standard deviation of the accumulation level values in the data set is large, adjusting the safety risk level of the feed to the high-risk level, and strengthening control measures; and
when the mean of the accumulation level values in the data set is less than the reference threshold of the mean, the standard deviation of the accumulation level values in the data set is less than the reference threshold of the standard deviation, and the mean of the accumulation level values in the data set and the standard deviation of the accumulation level values in the data set are low, keeping the safety risk level of the feed at the low-risk level, and continuing to conduct regular accumulation level monitoring.

In an embodiment, the disclosure further provides a system for assessing a safety risk level of a feed in cattle and sheep breeding, configured to implement the method. The system includes: a data acquisition module, a model prediction module, a risk assessment module, and a dynamic adjustment module.

The data acquisition module is configured to identify the different low-concentration chronic toxic substances in the feed for cattle and sheep through the sample testing and the data analysis, where the different low-concentration chronic toxic substances include the heavy metals, the mycotoxins, the antibiotic residues, and the environmental pollutants.

The model prediction module is configured to obtain, based on the different low-concentration chronic toxic substances, the biological enrichment abnormal index and the metabolic pathway deviation index of each of the different low-concentration chronic toxic substances, construct the accumulation effect model of the different low-concentration chronic toxic substances in the cattle and sheep bodies, and predict the accumulation levels of the different low-concentration chronic toxic substances after the intake by cattle and sheep.

The risk assessment module is configured to assess, according to the accumulation effect model, the potential health risks of the different low-concentration chronic toxic substances after the intake by cattle and sheep to obtain the assessment results; classify, according to the assessment results, the safety risk level of the feed as one of different risk levels including the high-risk level, the medium-risk level, and the low-risk level; and formulate the corresponding management measures and the feed usage restrictions for the different risk levels.

The dynamic adjustment module is configured to, when the safety risk level of the feed is classified as the medium-risk level, further analyze the potential health risks of the different low-concentration chronic toxic substances after the intake by cattle and sheep within the fixed time period to obtain the analysis results, and dynamically adjust, according to the analysis results, the safety risk level of the feed.

In an embodiment, each of the data acquisition module, the model prediction module, the risk assessment module, the dynamic adjustment module, the accumulation effect model, the dynamic metabolic network, the dynamic model of the toxic substance under normal metabolic conditions, and the abnormal metabolic model is embodied by at least one processor and at least one memory coupled to the at least one processor, and the at least one memory stores computer programs executable by the at least one processor.

In the above technical solution, the disclosure provides the following technical effects and advantages.

1. The disclosure introduces the assessment of the accumulation effect of chronic toxic substances, addressing the issue that existing feed safety assessment systems fail to consider the potential threat to the health of cattle and sheep caused by long-term exposure to low-concentration toxic substances. Through the sample testing and the data analysis, the low-concentration chronic toxic substances in feed, such as heavy metals, mycotoxins, and antibiotic residues, are identified. The disclosure constructs the accumulation effect model based on the biological enrichment abnormal index and the metabolic pathway deviation index to predict the long-term accumulation levels of these substances in cattle and sheep. The accumulation effect model, combined with machine learning technology, can accurately calculate the degree of accumulation of chronic toxic substances and assess health risks in real time.

2. Based on the assessment results of the accumulation effect model, the disclosure categorizes feed safety risks into three levels: high, medium, and low. Corresponding management measures and usage restrictions are formulated according to the different levels. Particularly in the assessment of the feed with the medium-risk level, the system can dynamically collect and analyze accumulation data to calculate the mean and the standard deviation of accumulation levels, thereby adjusting the safety risk level of the feed in real time. If the accumulation level has a small fluctuation and a high mean, the risk level will be upgraded to the high-risk level, and enhanced control measures will be implemented. If the accumulation level has a low mean and large fluctuations, the risk level will be dynamically adjusted to ensure timely warnings and appropriate management actions. This method can effectively prevent and control the long-term health hazards of chronic toxic substances in the feed for cattle and sheep, thereby enhancing the safety of breeding operations.

### BRIEF DESCRIPTION OF DRAWINGS

In order to more clearly illustrate the embodiments of the disclosure or the technical solutions in the related art, a brief introduction will be given to the drawings required for the embodiments. It is apparent that the drawings described below are only some embodiments recorded in the disclosure. For those skilled in the art, other drawings can also be obtained based on these drawings.

FIG. 1 illustrates a flowchart diagram of a method of the disclosure.

FIG. 2 illustrates a module diagram of a system of the disclosure.

### DETAILED DESCRIPTION OF EMBODIMENTS

In order to clarify the purpose, technical solution, and advantages of the embodiments of the disclosure, a clear and complete description of the technical solution in the embodiments of the disclosure is provided below in conjunction with the accompanying drawings. Apparently, the described embodiments are a part of the embodiments of the disclosure, not all of them. Based on the embodiments of the disclosure, all other embodiments obtained by those skilled in the art without creative labor are within the scope of protection of the disclosure.

### Embodiment 1

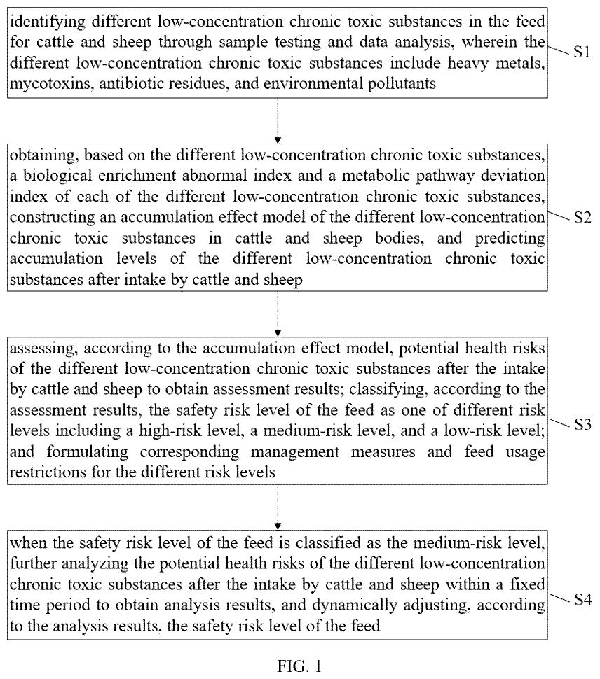

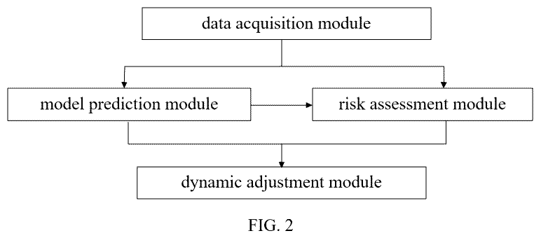

Referring to FIGS. 1 and 2, an embodiment of the disclosure provides a method for assessing a safety risk level of a feed in cattle and sheep breeding, including the following steps S1-S4:
S1, identifying different low-concentration chronic toxic substances in the feed for cattle and sheep through sample testing and data analysis, where the different low-concentration chronic toxic substances include heavy metals, mycotoxins, antibiotic residues, and environmental pollutants;
S2: obtaining, based on the different low-concentration chronic toxic substances, a biological enrichment abnormal index and a metabolic pathway deviation index of each of the different low-concentration chronic toxic substances, constructing an accumulation effect model of the different low-concentration chronic toxic substances in cattle and sheep bodies, and predicting accumulation levels of the different low-concentration chronic toxic substances after intake by cattle and sheep;
S3: assessing, according to the accumulation effect model, potential health risks of the different low-concentration chronic toxic substances after the intake by cattle and sheep to obtain assessment results; classifying, according to the assessment results, the safety risk level of the feed as one of different risk levels including a high-risk level, a medium-risk level, and a low-risk level; and formulating corresponding management measures and feed usage restrictions for the different risk levels; and
S4: when the safety risk level of the feed is classified as the medium-risk level, further analyzing the potential health risks of the different low-concentration chronic toxic substances after the intake by cattle and sheep within a fixed time period to obtain analysis results, and dynamically adjusting, according to the analysis results, the safety risk level of the feed.

In step S1, through the sample testing and the data analysis, the different low-concentration chronic toxic substances in the feed for cattle and sheep are identified, and the low-concentration chronic toxic substances include: the heavy metals, the mycotoxins, the antibiotic residues, and the environmental pollutants. Step S1 specifically includes the following steps.

In the safety assessment of the feed in cattle and sheep breeding, the low-concentration chronic toxic substances in the feed for cattle and sheep can be identified through the sample testing and the data analysis, and these toxic substances include but are not limited to the heavy metals, the mycotoxins, the antibiotic residues, and the environmental pollutants.

Heavy metals: The feed may contain trace amounts of the heavy metals, such as lead, cadmium, mercury, and arsenic. These heavy metals may not immediately affect the health of cattle and sheep at low concentrations. However, long-term intake can lead to their accumulation in the body, eventually causing liver and kidney damage and potentially affecting the animal's immune system.

Mycotoxins: Improper feed storage or transport can lead to mold contamination and the production of the mycotoxins, such as aflatoxins, zearalenone, and ochratoxins. Even at low concentrations, these mycotoxins can disrupt the digestive and reproductive systems of cattle and sheep after long-term accumulation, increasing infection risks.

Antibiotic residues: Due to the extensive use of antibiotics in animal husbandry, there may be small amounts of the antibiotic residues in the feed. Long-term low-dose antibiotic intake can lead to antibiotic resistance in pathogens in cattle and sheep, disrupt the balance of their gut microbiota, reduce the immunity, and ultimately cause a threat to human health through the spread of antibiotic-resistant bacteria.

Environmental pollutants: Some industrial pollutants, such as organic pollutants including polycyclic aromatic hydrocarbons (PAHs) and polychlorinated biphenyls (PCBs), may enter the feed chain through air, water, or soil. These environmental pollutants, even at low concentrations, have bioaccumulation properties. The long-term accumulation of the environmental pollutants in cattle and sheep can have potential chronic toxic effects on their health.

In the disclosure, through scientific sample testing and data analysis, these low-concentration chronic toxic substances can be identified and monitored, thereby providing data for feed safety risk grading and helps formulate management measures to reduce long-term health risks in cattle and sheep.

In step S2, based on the identified low-concentration chronic toxic substances, a biological enrichment abnormal index and a metabolic pathway deviation index of each of the different low-concentration chronic toxic substances are obtained, an accumulation effect model of the different low-concentration chronic toxic substances in cattle and sheep bodies is constructed, and accumulation levels of the different low-concentration chronic toxic substances after intake by cattle and sheep are predicted.

Based on the identified low-concentration chronic toxic substances, the biological enrichment abnormal index of each of the different low-concentration chronic toxic substances is obtained. The biological enrichment abnormal index is obtained through the following steps.

Factors affecting a biological enrichment process of a toxic substance of the different low-concentration chronic toxic substances in the cattle and sheep bodies are defined as nodes in a Bayesian network, where the factors include: an intake I, an absorption rate A, a metabolism rate M, and an excretion rate E, and a concentration accumulation level of the toxic substance is marked as C.

A conditional probability table for each of the nodes is set to describe a possible variable value and a probability of the variable value under a given parent node condition. For example, P(A|I) represents a possible value of the absorption rate A given the intake I. P(C|A,M,E) represents a possible value of the concentration accumulation level C under conditions of the absorption rate A, the metabolism rate M, and the excretion rate E. According to a structure of the Bayesian network and the conditional probability table, a marginal probability of the concentration accumulation level is solved through marginalization as follows: P(C)=Σ_{I} Σ_{A} Σ_{M} Σ_{E} P(C|A,M,E)·P(A|I)·P(I)·P(M)·P(E), where P(C) represents an accumulation probability distribution of the toxic substance in vivo. The biological enrichment abnormal index is an indicator to measure whether the accumulation level is beyond the normal range. A normal accumulation range is set as Cₙₒᵣₘₐₗ, and the biological enrichment abnormal index is calculated through the following formula: BA=P(C>Cₙₒᵣₘₐₗ)=1−Σ_{C≤C}_{ₙₒᵣₘₐₗ} P(C), where BA represents the biological enrichment abnormal index.

The higher the biological enrichment abnormal index, the more it indicates that the accumulation level of the chronic toxic substance in cattle and sheep significantly exceeds the normal range, indicating an abnormal enrichment situation. This implies that there is a deviation in the intake, absorption, metabolism, or excretion process of the toxic substance, leading to the continuous accumulation of the toxic substance in vivo. A high biological enrichment abnormal index suggests that cattle and sheep may face significant chronic health risks, which in long term may increase the possibility of organ damage, weakened immunity, chronic poisoning, and other problems. Such abnormal enrichment situations may require immediate management measures, such as adjusting feed components or controlling intake frequency, to reduce risks.

In contrast, the lower the biological enrichment abnormal index, the more it indicates that the accumulation level of the toxic substance in cattle and sheep is within the normal or lower range, and the accumulation process is stable and in line with normal physiological metabolic patterns. A low biological enrichment abnormal index suggests that the concentration of the toxic substance in cattle and sheep is within a safe range and has a smaller threat to health. This typically means that the toxic substance in the feed is effectively metabolized and excreted, without forming a risk of enrichment. The low biological enrichment abnormal index generally indicates that the feed safety level is higher and the feed can continue to be used without special intervention measures.

Based on the identified low-concentration chronic toxic substances, the metabolic pathway deviation index of each of the different low-concentration chronic toxic substances are obtained. The metabolic pathway deviation index is obtained through the following steps.

In a dynamic metabolic network model, a change in a concentration of a metabolite of metabolites Cᵢ(t) can usually be represented by a set of ordinary differential equations, and the equation is expressed as follows:

$$\frac{{dC}_{i} ( t )}{d t} = {f}_{i} ( {C}_{1} ( t ) , {C}_{2} ( t ) , … , {C}_{n} ( t ) , {k}_{i} ) ,$$

where Cᵢ(t) represents the concentration of the metabolite i at time t, fᵢ represents a rate equation of metabolic reaction, kᵢ represents a reaction rate constant, and n represents a total number of the metabolites. Each fᵢ is usually defined based on chemical reaction kinetics (such as the law of mass action). A dynamic model of the toxic substance under normal metabolic conditions is established. A normal metabolic parameter

$${k}_{i}^{normal}$$

and a corresponding normal metabolite concentration change

$${C}_{n}^{normal} ( t )$$

of the toxic substance are obtained through experimental data or literature. An equation for a normal metabolic pathway is as follows:

$$\frac{d {C}_{i}^{normal} ( t )}{d t} = {f}_{i} ( {C}_{1}^{normal} ( t ) , {C}_{2}^{normal} ( t ) , … , {C}_{n}^{normal} ( t ) , {k}_{i}^{normal} ) .$$

Under certain conditions (such as increased intake of the toxic substance or other environmental factors), a metabolic parameter

$${k}_{i}^{obs}$$

and a metabolite concentration change

$${C}_{n}^{obs} ( t )$$

of the toxic substance are re-measured. An abnormal metabolic model is established, and an equation for an abnormal metabolic pathway is as follows:

$$\frac{d {C}_{i}^{obs} ( t )}{d t} = {f}_{i} ( {C}_{1}^{obs} ( t ) , {C}_{2}^{obs} ( t ) , … , {C}_{n}^{obs} ( t ) , {k}_{i}^{obs} ) .$$

To quantify a deviation between the normal metabolic pathway and the abnormal metabolic pathway, a metabolic deviation Dᵢ(t) at the time t is calculated through the following formula:

$${D}_{i} ( t ) = \vert {C}_{i}^{obs} ( t ) - {C}_{i}^{normal} ( t ) \vert .$$

The metabolic pathway deviation index MP is an integral of all metabolite concentration deviations over a time period, and is calculated through the following formula:

$$MP = \frac{1}{n} {\sum }_{i = 1}^{n} {\int }_{0}^{T} {D}_{i} ( t ) dt ,$$

where T represents a total duration of an observation period, and n represents the total number of the metabolites.

The larger the metabolic pathway deviation index, the more significant the deviation in the metabolic process of the chronic toxic substance in cattle and sheep. Specifically, this indicates that the metabolic process is not proceeding along the normal physiological pathway, and instead, there may be excessive accumulation, toxic reactions, or abnormal transformations. For example, some toxic substances may impair the metabolic capacity of the liver or kidneys, preventing the effective elimination of toxic substances, thus leading to their accumulation in the body. Such situation typically increases the risk of chronic health issues, such as organ damage, immune system disorders, or even cancer. Therefore, a larger metabolic pathway deviation index usually implies a higher accumulation level of the chronic toxic substance in cattle and sheep, with an increased health risk.

In contrast, the smaller the metabolic pathway deviation index, the closer the metabolic process of the chronic toxic substance in cattle and sheep is to the normal physiological pattern, with the metabolite concentration becoming stable and showing no signs of abnormal enrichment. At this time, the toxic substance can be processed along the normal metabolic pathway and effectively eliminated through the excretory system. A smaller metabolic pathway deviation index means that the toxic substance is not accumulating significantly, and the metabolic process in vivo is in a healthy state of equilibrium. This indicates that the health risk for cattle and sheep is lower, and the likelihood of chronic poisoning or organ damage is small, hence the accumulation level of the chronic toxic substance is lower.

Based on the biological enrichment abnormal index and the metabolic pathway deviation index of each of the different low-concentration chronic toxic substances, the accumulation effect model of the different low-concentration chronic toxic substances in the cattle and sheep bodies is constructed, and the accumulation levels of the different low-concentration chronic toxic substances after the intake by cattle and sheep are predicted, where the accumulation effect model is a machine learning model.

The biological enrichment abnormal index and the metabolic pathway deviation index are converted into a comprehensive feature vector for each of the different low-concentration chronic toxic substances as an input of the machine learning model. With a predicted target being to predict, based on the comprehensive feature vector for each of the different low-concentration chronic toxic substances, accumulation level value labels of the different low-concentration chronic toxic substances after the intake by cattle and sheep, and a training target being to minimize a sum of prediction errors for the different low-concentration chronic toxic substances after the intake by cattle and sheep, the machine learning model is trained until the sum of the prediction errors reaches convergence. According to output results of the accumulation effect model, accumulation level values of the different low-concentration chronic toxic substances after the intake by cattle and sheep are determined, where the machine learning model is a polynomial regression model.

A method for obtaining the accumulation levels of the different chronic toxic substances after intake by cattle and sheep is as follows: From the training data of the comprehensive feature vector of the trained machine learning model, the corresponding functional expression is obtained: LR=F(BA,MP); where F represents the output function of the model, BA represents the biological enrichment abnormal index, MP represents the metabolic pathway deviation index, and LR represents the accumulation level of the chronic toxic substance after intake by cattle and sheep.

In step S3, according to the accumulation effect model, potential health risks of the different low-concentration chronic toxic substances after the intake by cattle and sheep are assessed to obtain assessment results; according to the assessment results, the safety risk level of the feed is classified as one of different risk levels including a high-risk level, a medium-risk level, and a low-risk level; and corresponding management measures and feed usage restrictions are formulated for the different risk levels. In an embodiment, the corresponding management measures and feed usage restrictions are performed according to the safety risk level of the feed.

The accumulation level values of the different low-concentration chronic toxic substances after the intake by cattle and sheep are compared with a gradient standard threshold, where the gradient standard threshold includes a first standard threshold and a second standard threshold, and the first standard threshold is less than the second standard threshold. The accumulation level values of the different low-concentration chronic toxic substances after the intake by cattle and sheep are compared with the first standard threshold and the second standard threshold respectively.

When one of the accumulation level values of the different low-concentration different chronic toxic substances after the intake by cattle and sheep is greater than the second standard threshold, which indicates that the potential health risks of the different low-concentration chronic toxic substances after the intake by cattle and sheep are high, a first-level warning signal is generated, the safety risk level of the feed is classified as the high-risk level, and restrictive measures are taken immediately. For example, the feed is prohibited to use or sent to processing facilities for cleaning.

When one of the accumulation level values of the different low-concentration chronic toxic substances after the intake by cattle and sheep is greater than or equal to the first standard threshold and less than or equal to the second standard threshold, which indicates that the potential health risks of the different low-concentration chronic toxic substances after the intake by cattle and sheep are medium, a second-level warning signal is generated, the safety risk level of the feed is classified as the medium-risk level, monitoring is strengthened, and a usage frequency is reduced to ensure the health of cattle and sheep and avoid long-term use.

When the accumulation level values of the different low-concentration chronic toxic substances after the intake by cattle and sheep are less than the first standard threshold, which indicates that the potential health risks of the different low-concentration chronic toxic substances after the intake by cattle and sheep are low, a third-level warning signal is generated, the safety risk level of the feed is classified as the low-risk level, and the feed can continue to be used but should be regularly checked to ensure it does not exceed the safety threshold.

It should be noted here that the importance of the first-level warning signal is greater than the importance of the second-level warning signal, and the importance of the second-level warning signal is greater than the importance of the third-level warning signal. Relevant personnel can take the corresponding measures according to different warning signal levels.

In step S4, when the safety risk level of the feed is classified as the medium-risk level, the potential health risks of the different low-concentration chronic toxic substances after the intake by cattle and sheep within a fixed time period are further analyzed to obtain analysis results, and the safety risk level of the feed is dynamically adjusted according to the analysis results.

When the safety risk level of the feed is classified as the medium-risk level, that is, one of the accumulation level values of the different low-concentration chronic toxic substances after the intake by cattle and sheep generated within the fixed time period is greater than or equal to the first standard threshold and less than or equal to the second standard threshold, accumulation level values of the different low-concentration chronic toxic substances after the intake by cattle and sheep generated in a subsequent fixed time period that are greater than or equal to the first standard threshold and less than or equal to the second standard threshold are collected, a data set is established correspondingly, a mean and a standard deviation of the accumulation level values in the data set are calculated, the potential health risks of the different low-concentration chronic toxic substances after the intake by cattle and sheep are further analyzed to obtain the analysis results, and the safety risk level of the feed is dynamically adjusted according to the analysis results.

When the mean of the accumulation level values in the data set is greater than or equal to a reference threshold of the mean, the standard deviation of the accumulation level values in the data set is less than a reference threshold of the standard deviation, and the mean of the accumulation level values in the data set is high with small fluctuations, indicating a higher and more stable potential health risk for cattle and sheep, it may be considered to upgrade the safety risk level of the feed to the high-risk level, and take corresponding management measures, such as strengthening monitoring or restricting the use of the feed.

When the mean of the accumulation level values in the data set is greater than or equal to the reference threshold of the mean, the standard deviation of the accumulation level values in the data set is greater than or equal to the reference threshold of the standard deviation, and the mean of the accumulation level values in the data set is high but the standard deviation of the accumulation level values in the data set is large, it indicates that although the accumulation level is high, there may be significant fluctuations over different time periods. This could imply that the risk is relatively unstable, the feed should be re-assessed, and it may be considered to maintain temporarily the safety risk level of the feed at the medium-risk level, and strengthen monitoring of accumulation fluctuation.

When the mean of the accumulation level values in the data set is less than the reference threshold of the mean, the standard deviation of the accumulation level values in the data set is greater than or equal to the reference threshold of the standard deviation, and the mean of the accumulation level values in the data set is low but the standard deviation of the accumulation level values in the data set is large, it indicates that the accumulation level is relatively low, but there is significant fluctuation. This could lead to sudden increases in the accumulation levels at certain times, posing potential risks. In this case, it is recommended to further analyze the causes of the fluctuations, the safety risk level of the feed may be adjusted to the high-risk level, and control measures are strengthened.

When the mean of the accumulation level values in the data set is less than the reference threshold of the mean, the standard deviation of the accumulation level values in the data set is less than the reference threshold of the standard deviation, and the mean of the accumulation level values in the data set and the standard deviation of the accumulation level values in the data set are low, it indicates that the accumulation levels of the chronic toxic substances are low and stable. This generally implies a lower health risk. It may be considered to keep the safety risk level of the feed at the low-risk level, and continue to conduct regular accumulation level monitoring.

In this embodiment, the low-concentration chronic toxic substances in the feed (such as the heavy metals, the mycotoxins, the antibiotic residues, and the environmental pollutants) are identified through the sample testing and the data analysis. The accumulation effect model is constructed based on these substances to predict their accumulation levels in cattle and sheep. The potential health risks of the chronic toxic substances to cattle and sheep are assessed by calculating the biological enrichment abnormal index and the metabolic pathway deviation index. According to the assessment results, the safety risk of the feed is categorized into one of three levels: high, medium, and low, and corresponding management measures and usage restrictions are formulated and performed. When the safety risk level of the feed is medium, further analysis of the accumulation levels of the chronic toxic substances within a fixed time period is conducted, and the risk level is dynamically adjusted to ensure the safety and health protection of feed use.

### Embodiment 2

An embodiment provides a system for assessing a safety risk level of a feed in cattle and sheep breeding, including: a data acquisition module, a model prediction module, a risk assessment module, and a dynamic adjustment module.

The data acquisition module is configured to identify different low-concentration chronic toxic substances in the feed for cattle and sheep through sample testing and data analysis, where the different low-concentration chronic toxic substances include heavy metals, mycotoxins, antibiotic residues, and environmental pollutants.

The model prediction module is configured to obtain, based on the different low-concentration chronic toxic substances, a biological enrichment abnormal index and a metabolic pathway deviation index of each of the different low-concentration chronic toxic substances, construct an accumulation effect model of the different low-concentration chronic toxic substances in cattle and sheep bodies, and predict accumulation levels of the different low-concentration chronic toxic substances after intake by cattle and sheep.

The risk assessment module is configured to assess, according to the accumulation effect model, potential health risks of the different low-concentration chronic toxic substances after the intake by cattle and sheep to obtain assessment results; classify, according to the assessment results, the safety risk level of the feed as one of different risk levels comprising a high-risk level, a medium-risk level, and a low-risk level; and formulate corresponding management measures and feed usage restrictions for the different risk levels.

The dynamic adjustment module is configured to, when the safety risk level of the feed is classified as the medium-risk level, further analyze the potential health risks of the different low-concentration chronic toxic substances after the intake by cattle and sheep within a fixed time period to obtain analysis results, and dynamically adjust, according to the analysis results, the safety risk level of the feed.

The aforementioned formulas are calculated by removing dimensions to obtain their numerical values. The formulas are derived from extensive data collection and software simulation to approximate the most realistic situation. The preset parameters within the formulas are set by those skilled in the art based on actual conditions.

It should be understood that the term “and/or” herein is only a description of the association relationship between related objects, indicating that there can be three types of relationships, for example, A and/or B, which can represent: the existence of A alone, the existence of A and B at the same time, and the existence of B alone, where A and B can be single or complex numbers. In addition, the character “/” herein generally indicates a “or” relationship between related objects, but it may also indicate a “and/or” relationship, which can be understood by referring to the context.

Those skilled in the art can realize that the units and algorithm steps described in the embodiments disclosed herein can be implemented through electronic hardware, or a combination of computer software and electronic hardware. Whether these functions are executed in hardware or software depends on the specific application and design constraints of the technical solution. Professional technicians can use different methods to achieve the described functions for each specific application, but such implementation should not be considered beyond the scope of the disclosure.

The above is only a specific implementation of the disclosure, but the scope of protection of the disclosure is not limited to this. Those skilled in the art can easily think of changes or replacements within the technical scope disclosed in the disclosure, which should be included in the scope of protection of the disclosure.

What is claimed is:

1. A method for assessing a safety risk level of a feed in cattle and sheep breeding, comprising the following steps:
S1, identifying different low-concentration chronic toxic substances in the feed for cattle and sheep through sample testing and data analysis, wherein the different low-concentration chronic toxic substances comprise heavy metals, mycotoxins, antibiotic residues, and environmental pollutants;
S2: obtaining, based on the different low-concentration chronic toxic substances, a biological enrichment abnormal index and a metabolic pathway deviation index of each of the different low-concentration chronic toxic substances, constructing an accumulation effect model of the different low-concentration chronic toxic substances in cattle and sheep bodies, and predicting accumulation levels of the different low-concentration chronic toxic substances after intake by cattle and sheep;
wherein the obtaining, based on the different low-concentration chronic toxic substances, a biological enrichment abnormal index and a metabolic pathway deviation index of each of the different low-concentration chronic toxic substances comprises:
obtaining the biological enrichment abnormal index, comprising: defining factors affecting a biological enrichment process of a toxic substance of the different low-concentration chronic toxic substances in the cattle and sheep bodies as nodes in a Bayesian network, wherein the factors comprise: an intake I, an absorption rate A, a metabolism rate M, and an excretion rate E, and marking a concentration accumulation level of the toxic substance as C; setting a conditional probability table for each of the nodes to describe a variable value and a probability of the variable value under a given parent node condition; solving, according to a structure of the Bayesian network and the conditional probability table, a marginal probability of the concentration accumulation level through marginalization as follows: P(C)=Σ_{I} Σ_{A} Σ_{M} Σ_{E} P(C|A,M,E)·P(A|I)·P(I)·P(M)·P(E), where P(C) represents an accumulation probability distribution of the toxic substance in vivo; and setting a normal accumulation range as Cₙₒᵣₘₐₗ, and calculating the biological enrichment abnormal index through the following formula: BA=P(C>Cₙₒᵣₘₐₗ)=1−Σ_{C≤C}_{ₙₒᵣₘₐₗ} P(C), where BA represents the biological enrichment abnormal index; and
obtaining the metabolic pathway deviation index, comprising: in a dynamic metabolic network model, calculating a change in a concentration of a metabolite of metabolites Cᵢ(t) through the following formula:
$$\frac{d {C}_{i} ( t )}{d t} = {f}_{i} ( {C}_{1} ( t ) , {C}_{2} ( t ) , … , {C}_{n} ( t ) , {k}_{i} ) ,$$
where Cᵢ(t) represents the concentration of the metabolite i at time t, fᵢ represents a rate equation of metabolic reaction and is defined based on chemical reaction kinetics, kᵢ represents a reaction rate constant, and n represents a total number of the metabolites; establishing a dynamic model of the toxic substance under normal metabolic conditions, obtaining a normal metabolic parameter
$${k}_{i}^{normal}$$
and a corresponding normal metabolite concentration change
$${C}_{n}^{normal} ( t )$$
of the toxic substance, and obtaining an equation for a normal metabolic pathway as follows:
$$\frac{d {C}_{i}^{normal} ( t )}{d t} = {f}_{i} ( {C}_{1}^{normal} ( t ) , {C}_{2}^{normal} ( t ) , … , {C}_{n}^{normal} ( t ) , {k}_{i}^{normal} ) ;$$
re-measuring a metabolic parameter
$${k}_{i}^{obs}$$
and a metabolite concentration change
$${C}_{n}^{obs} ( t )$$
of the toxic substance, establishing an abnormal metabolic model, and obtaining an equation for an abnormal metabolic pathway as follows:
$$\frac{d {C}_{i}^{obs} ( t )}{d t} = {f}_{i} ( {C}_{1}^{obs} ( t ) , {C}_{2}^{obs} ( t ) , … , {C}_{n}^{obs} ( t ) , {k}_{i}^{obs} ) ;$$
calculating a metabolic deviation Dᵢ(t) at the time t through the following formula to quantify a deviation between the normal metabolic pathway and the abnormal metabolic pathway:
$${D}_{i} ( t ) = \vert {C}_{i}^{obs} ( t ) - {C}_{i}^{normal} ( t ) \vert ;$$
and calculating the metabolic pathway deviation index MP being an integral of metabolite concentration deviations over a time period through the following formula:
$$MP = \frac{1}{n} {\sum }_{i = 1}^{n} {\int }_{0}^{T} {D}_{i} ( t ) d t ,$$
where T represents a total duration of an observation period, and n represents the total number of the metabolites;
S3: assessing, according to the accumulation effect model, potential health risks of the different low-concentration chronic toxic substances after the intake by cattle and sheep to obtain assessment results; classifying, according to the assessment results, the safety risk level of the feed as one of different risk levels comprising a high-risk level, a medium-risk level, and a low-risk level; and formulating corresponding management measures and feed usage restrictions for the different risk levels; and
S4: when the safety risk level of the feed is classified as the medium-risk level, further analyzing the potential health risks of the different low-concentration chronic toxic substances after the intake by cattle and sheep within a fixed time period to obtain analysis results, and dynamically adjusting, according to the analysis results, the safety risk level of the feed.

2. The method for assessing the safety risk level of the feed in the cattle and sheep breeding as claimed in claim 1, wherein step S2 further comprises: constructing, based on the biological enrichment abnormal index and the metabolic pathway deviation index of each of the different low-concentration chronic toxic substances, the accumulation effect model of the different low-concentration chronic toxic substances in the cattle and sheep bodies, and predicting the accumulation levels of the different low-concentration chronic toxic substances after the intake by cattle and sheep, wherein the accumulation effect model is a machine learning model, specifically comprising:
converting the biological enrichment abnormal index and the metabolic pathway deviation index into a comprehensive feature vector for each of the different low-concentration chronic toxic substances as an input of the machine learning model;
training the machine learning model with a predicted target being to predict, based on the comprehensive feature vector for each of the different low-concentration chronic toxic substances, accumulation level value labels of the different low-concentration chronic toxic substances after the intake by cattle and sheep, and a training target being to minimize a sum of prediction errors for the different low-concentration chronic toxic substances after the intake by cattle and sheep until the sum of the prediction errors reaches convergence; and
determining, according to model output results, accumulation level values of the different low-concentration chronic toxic substances after the intake by cattle and sheep, wherein the machine learning model is a polynomial regression model.

3. The method for assessing the safety risk level of the feed in the cattle and sheep breeding as claimed in claim 2, wherein step S3 specifically comprises:
comparing the accumulation level values of the different low-concentration chronic toxic substances after the intake by cattle and sheep with a gradient standard threshold, wherein the gradient standard threshold comprises a first standard threshold and a second standard threshold, and the first standard threshold is less than the second standard threshold, specifically comprising:
comparing the accumulation level values of the different low-concentration chronic toxic substances after the intake by cattle and sheep with the first standard threshold and the second standard threshold respectively;
when one of the accumulation level values of the different low-concentration different chronic toxic substances after the intake by cattle and sheep is greater than the second standard threshold, which indicates that the potential health risks of the different low-concentration chronic toxic substances after the intake by cattle and sheep are high, generating a first-level warning signal, classifying the safety risk level of the feed as the high-risk level, and taking restrictive measures immediately;
when one of the accumulation level values of the different low-concentration chronic toxic substances after the intake by cattle and sheep is greater than or equal to the first standard threshold and less than or equal to the second standard threshold, which indicates that the potential health risks of the different low-concentration chronic toxic substances after the intake by cattle and sheep are medium, generating a second-level warning signal, classifying the safety risk level of the feed as the medium-risk level, strengthening monitoring, and reducing a usage frequency;
when the accumulation level values of the different low-concentration chronic toxic substances after the intake by cattle and sheep are less than the first standard threshold, which indicates that the potential health risks of the different low-concentration chronic toxic substances after the intake by cattle and sheep are low, generating a third-level warning signal, classifying the safety risk level of the feed as the low-risk level, and continuing to use the feed with regular checks.

4. The method for assessing the safety risk level of the feed in the cattle and sheep breeding as claimed in claim 3, wherein step S4 specifically comprises:
when the safety risk level of the feed is classified as the medium-risk level, and one of the accumulation level values of the different low-concentration chronic toxic substances after the intake by cattle and sheep generated within the fixed time period is greater than or equal to the first standard threshold and less than or equal to the second standard threshold, collecting accumulation level values of the different low-concentration chronic toxic substances after the intake by cattle and sheep generated in a subsequent fixed time period that are greater than or equal to the first standard threshold and less than or equal to the second standard threshold, establishing a data set correspondingly, calculating a mean and a standard deviation of the accumulation level values in the data set, further analyzing the potential health risks of the different low-concentration chronic toxic substances after the intake by cattle and sheep to obtain the analysis results, and dynamically adjusting, according to the analysis results, the safety risk level of the feed.

5. The method for assessing the safety risk level of the feed in the cattle and sheep breeding as claimed in claim 4, wherein the dynamically adjusting, according to the analysis results, the safety risk level of the feed comprises:
when the mean of the accumulation level values in the data set is greater than or equal to a reference threshold of the mean, the standard deviation of the accumulation level values in the data set is less than a reference threshold of the standard deviation, and the mean of the accumulation level values in the data set is high with small fluctuations, upgrading the safety risk level of the feed to the high-risk level, and taking corresponding management measures;
when the mean of the accumulation level values in the data set is greater than or equal to the reference threshold of the mean, the standard deviation of the accumulation level values in the data set is greater than or equal to the reference threshold of the standard deviation, and the mean of the accumulation level values in the data set is high but the standard deviation of the accumulation level values in the data set is large, re-assessing the feed, maintaining temporarily the safety risk level of the feed at the medium-risk level, and strengthening monitoring of accumulation fluctuation;
when the mean of the accumulation level values in the data set is less than the reference threshold of the mean, the standard deviation of the accumulation level values in the data set is greater than or equal to the reference threshold of the standard deviation, and the mean of the accumulation level values in the data set is low but the standard deviation of the accumulation level values in the data set is large, adjusting the safety risk level of the feed to the high-risk level, and strengthening control measures; and
when the mean of the accumulation level values in the data set is less than the reference threshold of the mean, the standard deviation of the accumulation level values in the data set is less than the reference threshold of the standard deviation, and the mean of the accumulation level values in the data set and the standard deviation of the accumulation level values in the data set are low, keeping the safety risk level of the feed at the low-risk level, and continuing to conduct regular accumulation level monitoring.

6. A system for assessing a safety risk level of a feed in cattle and sheep breeding, configured to implement the method as claimed in claim 1, wherein the system comprises: a data acquisition module, a model prediction module, a risk assessment module, and a dynamic adjustment module;
the data acquisition module is configured to identify the different low-concentration chronic toxic substances in the feed for cattle and sheep through the sample testing and the data analysis, wherein the different low-concentration chronic toxic substances comprise the heavy metals, the mycotoxins, the antibiotic residues, and the environmental pollutants;
the model prediction module is configured to obtain, based on the different low-concentration chronic toxic substances, the biological enrichment abnormal index and the metabolic pathway deviation index of each of the different low-concentration chronic toxic substances, construct the accumulation effect model of the different low-concentration chronic toxic substances in the cattle and sheep bodies, and predict the accumulation levels of the different low-concentration chronic toxic substances after the intake by cattle and sheep;
the risk assessment module is configured to assess, according to the accumulation effect model, the potential health risks of the different low-concentration chronic toxic substances after the intake by cattle and sheep to obtain the assessment results; classify, according to the assessment results, the safety risk level of the feed as one of different risk levels comprising the high-risk level, the medium-risk level, and the low-risk level; and formulate the corresponding management measures and the feed usage restrictions for the different risk levels; and
the dynamic adjustment module is configured to, when the safety risk level of the feed is classified as the medium-risk level, further analyze the potential health risks of the different low-concentration chronic toxic substances after the intake by cattle and sheep within the fixed time period to obtain the analysis results, and dynamically adjust, according to the analysis results, the safety risk level of the feed.
